(19) United States
(12) Patent Application Publication
(10) Pub. No.: US 2024/0385163 A1
(43) Pub. Date: Nov. 21, 2024

(54) STIFFNESS AND STRENGTH ASSESSMENT OF GROWING PLANT MATERIAL

(71) Applicant: Brigham Young University, Provo, UT (US)

(72) Inventors: Kirsten Steele, Provo, UT (US); Jordan Porter, Provo, UT (US); Douglas Cook, Fountain Green, UT (US)

(21) Appl. No.: 18/576,639

(22) PCT Filed: Jul. 13, 2022
(86) PCT No.: PCT/US2022/073689

§ 371 Date: Jan. 4, 2024

Related U.S. Application Data
(60) Provisional application No. 63/203,225, filed on Jul. 13, 2021.

(51) Int. Cl. G01N 33/00; A01B 76/00; G01N 3/02

(52) U.S. Cl. CPC G01N 33/0098; A01B 76/00; G01N 3/02

(57) ABSTRACT

In a general aspect, an apparatus configured for attachment to a vehicle can include: an alignment mechanism configured, as result of movement of the vehicle relative to a plant stalk, to place the plant stalk in a known position relative to the apparatus; a deflection mechanism configured, as result of at least one of the movement of the vehicle relative to the plant stalk or rotational movement of the deflection mechanism, to move the plant stalk from the known position to a deflected position, the deflected position being a distance from the known position; and a force sensor configured to determine an amount of force applied to move the plant stalk from the known position to the deflected position.

### CROSS-REFERENCE TO RELATED APPLICATION

This application claims benefit of, and priority to, U.S. Provisional Patent Application No. 63/203,225, filed on Jul. 13, 2021, entitled “High-Throughput Measurement of Grain Stem Stiffness and Strength”, the disclosure of which is incorporated by reference herein in its entirety.

### TECHNICAL FIELD

This disclosure relates to structural assessment of crop material. More specifically, this disclosure relates to assessment of flexural stiffness and/or bending strength (e.g., breaking strength) of plant material.

### BACKGROUND

Agricultural crops are susceptible to damage from environmental conditions, such as weather. One type of such damage that can occur with stalked crops, such as grain or other crops, is lodging. Lodging is a state of permanent displacement of the stems or stalks from their upright position. It can be induced by external forces such as wind, rain or hail. Lodging that occurs in the root is referred to as root lodging and similarly lodging in a plant stalk (or stem) is referred to as stalk lodging. Stalk lodging occurs in two main types. Greensnap occurs during periods of rapid growth. Because greensnap typically occurs before pollination, it leads to a complete loss of grain for affected plants. Late-season stalk lodging occurs as the grain is drying down and is most severe just before harvest.

Gaining an understanding of how lodging occurs can be useful in the development of plant breeds that are more resistant to the occurrence of lodging. Two important physical attributes in understanding how lodging occurs in susceptible plant are bending strength and flexural stiffness. Breaking strength is a measure of an amount of bending load needed to break a plant's stalk. Flexural stiffness, which can be correlated with breaking strength, is a measure of a stem's ability to resist bending loads. Measuring bending strength is destructive to the measured plant, while measuring flexural stiffness is non-destructive.

Obtaining a large amount of flexural stiffness and/or bending strength data over a population of plants (e.g. at various stages of growth) can be useful for performing detailed statistical analyses and/or developing mathematical models (e.g., finite elements models) to gain a better understanding of how lodging occurs and how to reduce its occurrence. However, current approaches for obtaining such flexural stiffness and/or bending strength measurements are both labor intensive and time consuming.

### SUMMARY

In some aspects, the techniques described herein relate to an apparatus configured for attachment to a vehicle, the apparatus including: an alignment mechanism configured, as result of movement of the vehicle relative to a plant stalk, to place the plant stalk in a known position relative to the apparatus; a deflection mechanism configured, as result of at least one of the movement of the vehicle relative to the plant stalk or rotational movement of the deflection mechanism, to move the plant stalk from the known position to a deflected position, the deflected position being a distance from the known position; and a force sensor configured to determine an amount of force applied to move the plant stalk from the known position to the deflected position.

In some aspects, the techniques described herein relate to an apparatus, wherein the apparatus is configured such that a height of the apparatus from a growing surface of the plant stalk is adjustable.

In some aspects, the techniques described herein relate to an apparatus, wherein the force sensor includes a load cell configured to provide an electrical signal indicating the amount of force applied.

In some aspects, the techniques described herein relate to an apparatus, wherein the deflection mechanism includes a wheel including a plurality of radial vanes uniformly disposed around a perimeter of the wheel.

In some aspects, the techniques described herein relate to an apparatus, wherein the force sensor is operationally coupled with the wheel.

In some aspects, the techniques described herein relate to an apparatus, wherein the wheel is motorized and configured to rotate at a rotational speed corresponding with a velocity of the vehicle.

In some aspects, the techniques described herein relate to an apparatus, wherein: the plurality of radial vanes includes four vanes forming a cross shape; and the wheel is configured to: be rotationally fixed when the amount of force applied determined by the force sensor is below a threshold value; and in response to the amount of force applied determined by the force sensor reaching or exceeding the threshold value, rotate ninety degrees.

In some aspects, the techniques described herein relate to an apparatus, wherein the alignment mechanism includes at least one rigid member.

In some aspects, the techniques described herein relate to an apparatus, wherein: the deflection mechanism includes: a cantilever beam having a proximal end coupled to an end of a rigid member of the at least one rigid member in a fixed position relative to the rigid beam, a distal end of the cantilever beam being free moving; and the force sensor includes: a first strain gauge disposed on the cantilever beam at a first distance from the proximal end; and a second strain gauge disposed on the cantilever beam at a second distance from the proximal end, the second distance being greater than the first distance.

In some aspects, the techniques described herein relate to an apparatus, wherein: the first strain gauge is disposed on a first surface of the cantilever beam; and the second strain gauge is disposed on a second surface of the cantilever beam, the second surface being opposite the first surface.

In some aspects, the techniques described herein relate to an apparatus, wherein the first strain gauge and the second strain gauge are disposed on a same surface of the cantilever beam.

In some aspects, the techniques described herein relate to an apparatus, further including at least one additional strain gauge disposed on the cantilever beam, a third strain gauge of the at least one additional strain gauge being disposed at a third distance from the proximal end, the third distance being greater than or equal to the first distance.

In some aspects, the techniques described herein relate to an apparatus, wherein: the deflection mechanism includes: a cantilever beam having a proximal end coupled to an end of a rigid member of the at least one rigid members via a torsional spring, a distal end of the cantilever beam being free moving; and the force sensor includes: a torsional load cell operationally coupled with the torsional spring; a strain gauge disposed on the cantilever beam at a distance from the proximal end.

In some aspects, the techniques described herein relate to an apparatus, wherein the strain gauge is a first strain gauge and the distance is a first distance, the force sensor further including a second strain gauge disposed on the cantilever beam at a second distance from the proximal end, the second distance being greater than the first distance.

In some aspects, the techniques described herein relate to an apparatus, wherein: the deflection mechanism includes: a first wheel of a first radius being rotationally mounted on a first axis, the first wheel defining a first rotational plane; and a second wheel of a second radius being rotationally mounted on a second axis, the second wheel defining a second rotational plane that different from the first rotational plane; and a third wheel of the first radius being rotationally mounted on the first axis, the third wheel defining a third rotational plane that is different from the first rotational plane and the second rotational plane, the second axis being spaced from and parallel to the first axis, the first axis and the second axis being substantially parallel to a longitudinal axis of the plant stalk, the second rotational plane being disposed between the first rotational plane and the third rotational plane, and the first radius plus the second radius being greater than the spacing between the first axis and the second axis; and the force sensor is operatively coupled with one of the first wheel, the second wheel, or the third wheel.

In some aspects, the techniques described herein relate to an apparatus, wherein first wheel, the second wheel and the third wheel are rotationally motorized such that the second wheel counter-rotates with respect to the first wheel and third wheel.

In some aspects, the techniques described herein relate to an apparatus, wherein the force sensor is a first force sensor operationally coupled with the second wheel, the apparatus further including: a second force sensor operationally coupled with the first wheel.

In some aspects, the techniques described herein relate to an apparatus, further including a third force sensor operatively coupled with the third wheel.

In some aspects, the techniques described herein relate to an apparatus, wherein the alignment mechanism includes a linear bearing assembly configured to allow the deflection mechanism to move laterally along a line that is orthogonal to a direction of travel of the vehicle.

In some aspects, the techniques described herein relate to a method including: driving a vehicle having a measurement apparatus attached thereto proximate to a growing plant stalk; as a result of movement of the vehicle, an alignment mechanism placing a plant stalk in a known position relative to the measurement apparatus; moving with a deflection mechanism, as a result of at least one of the movement of the vehicle relative to the plant stalk or rotational movement of the deflection mechanism, the plant stalk from the known position to a deflected position, the deflected position being a distance from the known position; and determining, with a force sensor, an amount of force applied to move the plant stalk from the known position to the deflected position, the alignment mechanism, the deflection mechanism and the force sensor being included in the measurement apparatus.

In some aspects, the techniques described herein relate to a method, wherein determining the amount of force applied includes at least one of: receiving an electric signal from at least one load cell; or receiving an electric signal from at least one strain gauge.

### BRIEF DESCRIPTION OF THE DRAWINGS

FIG. 1A is a block diagram illustrating an example arrangement of a plurality of measurement apparatuses for assessment of flexural stiffness and/or bending strength of growing plant material.

FIG. 1B is a diagram that schematically illustrates an implementation of the example arrangement of FIG. 1A.

FIG. 2 is a block diagram schematically illustrating an example measurement apparatus that can be included in the implementations of FIGS. 1A and 1B.

FIGS. 3A and 3B are diagrams illustrating an example implementation of the measurement apparatus of FIG. 2.

FIGS. 4A and 4B are diagrams illustrating another example implementation of the measurement apparatus of FIG. 2.

FIGS. 5A and 5B are diagrams are diagrams illustrating an example cantilever beam sensor that can be included in the measurement apparatus of FIGS. 4A and 4B.

FIG. 6 is a diagram illustrating assessment of flexural stiffness and/or bending strength of growing plant material with an implementation of the measurement apparatus of FIGS. 4A and 4B.

FIG. 7 is a graph illustrating a force deformation curve corresponding with the assessment of FIG. 6.

FIGS. 8A and 8B are diagrams illustrating yet another example implementation of the measurement apparatus of FIG. 2.

FIGS. 9A and 9B are diagrams illustrating still another example implementation of the measurement apparatus of FIG. 2.

FIG. 10 is a flowchart illustrating an example method for assessment of flexural stiffness and/or bending strength of growing plant material.

Like reference symbols in the various drawings indicate like and/or similar elements. The drawings are for purposes of illustration and may not necessarily be to scale. Also, in some views, one or more features of an implementation may be obscured or omitted.

### DETAILED DESCRIPTION

This disclosure is directed to approaches for assessment of flexural stiffness and/or bending strength (e.g., breaking strength) of growing plant material, such as stalked plants or crops, which can include grain plants, or other crops such as sunflowers, etc. The approaches described herein can be used to automate assessment of flexural stiffness and/or bending strength of growing plant material, which can significantly increase assessment throughput as compared to previous (e.g., manual) approaches for performing such structural measurements, e.g. by one to two orders of magnitude. The approaches described herein can be useful in understanding how lodging (e.g., greensnap) occurs, by evaluation of physical characteristics of susceptible crop material, which can be useful to inform the development of plant breeds that are more resistant to such damage.

For purposes of illustration, the approaches described herein are generally discussed with respect to assessment of maize, where a given measurement apparatus acquires structural measurements for a single maize stalk at a time. However, these approaches can be similarly applied for assessment of other plants or crops, such as wheat, barley, oats, sorghum, sunflowers, etc. Depending on the particular implementation, as well as the particular crop on which structural measurements of flexural stiffness and/or bending strength are being made, plants can be assessed (measured) individually, or can be assessed using aggregate measurements, e.g., where structural measurements are obtained by a single measurement apparatus on multiple plants contemporaneously. For instance, in the example described herein, a measurement apparatus can be configured to acquire aggregate structural measurements for a plurality of plant stalks, including maize stalks.

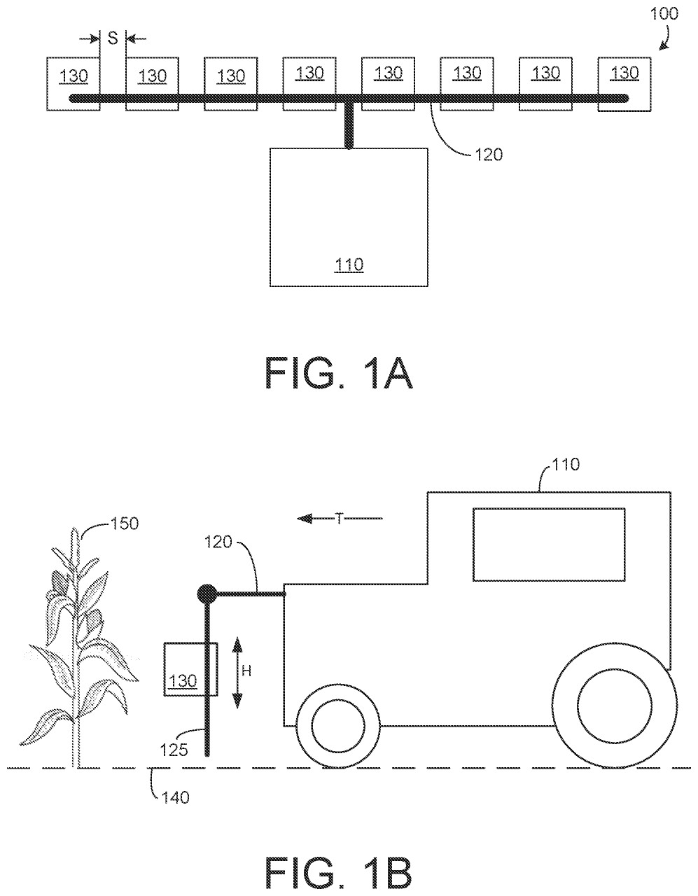

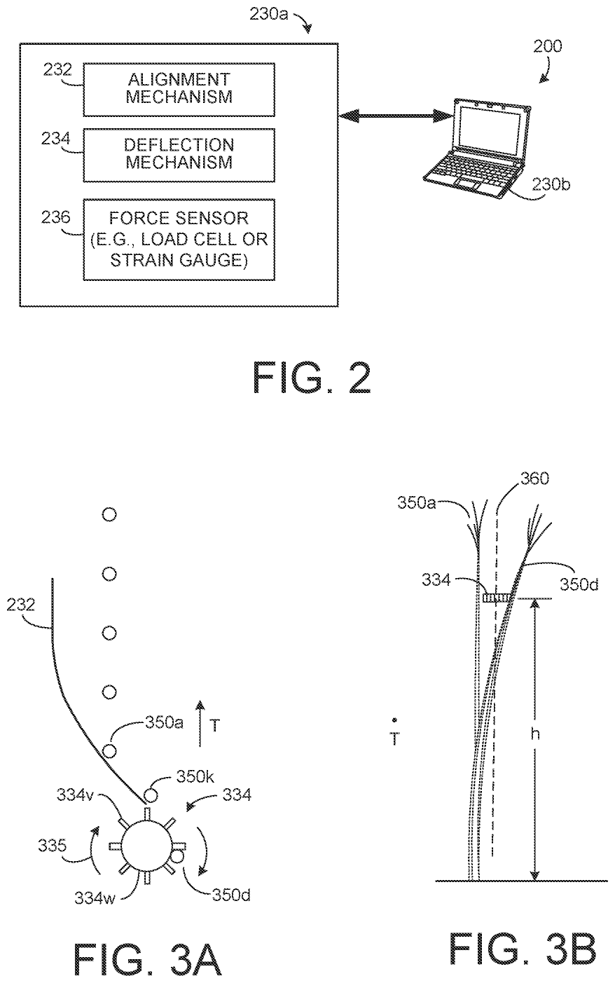

FIG. 1A is a block diagram illustrating an example arrangement 100 of a plurality of measurement apparatus 130 for automated assessment of flexural stiffness and/or bending strength of growing plant material, such as for assessing plants of a crop growing in a field. As shown in FIG. 1, the arrangement 100 includes a vehicle 110, such as an agricultural tractor (e.g., a maize detasseling tractor). A boom 120 is attached to (coupled with) the vehicle 110, and a plurality of measurement apparatus 130 are attached to (coupled with) the boom 120. The measurement apparatus 130 of FIG. 1 can be implemented using the example measurement apparatus described herein, such as those described with respect to FIGS. 2-9B. In some implementations, each of the measurement apparatus 130 can be a same measurement apparatus implementation, while in other implementations, different measurement apparatus implementations can be used for the measurement apparatus 130 of the arrangement 100.

In this example, eight measurement apparatus 130 are shown. In some implementations, fewer or more measurement apparatus 130 can be included in an arrangement such as the arrangement 100. For instance, a shorter boom could be used, and fewer measurement apparatus 130 could be included, or a longer boom could be used and more measurement apparatus 130 could be included. In the arrangement 100, a spacing S between adjacent measurement apparatus 130 will depend, at least in part, on a crop being assessed. For instance, the spacing S may be determined based on a separation distance between planting rows of the crop being assessed.

Using an arrangement, such as the arrangement 100, collection of structural measurements for assessment of flexural stiffness and/or bending strength of growing plant material can be automated, with such measurements for multiple planting rows of a crop being acquired in parallel (e.g., based on a number of measurement apparatus 130 included on the boom 120) as the vehicle is driven through a planting field including the crop. As noted above, such approaches can significantly improve throughput of obtaining such measurements over previous approaches.

FIG. 1B is a diagram that schematically illustrates an implementation of the example arrangement 100 of FIG. 1A. As shown in FIG. 1B, the vehicle 110 can be driven through a planting field 140 in a direction of travel T. In this example, as the vehicle 110 moves through the planting field 140, the measurement apparatus 130 can interface with plants in the field to obtain structural measurements for assessing flexural stiffness and/or bending strength of the plants. In this example, a maize plant 150 is shown for purposes of illustration.

As shown in FIG. 1B, the measurement apparatus 130 can be attached to (coupled with) a mounting pole 125 and a height of the measurement apparatus 130 can be adjusted by moving the measurement apparatus 130 up or down the mounting pole 125 along the line H, which provides for obtaining structural measurements at different vertical locations of plants being assessed. In some implementations, such height adjustment can be done manually, e.g., using a mechanically releasable collar that can be moved along the mounting pole 125. In other implementations, such height adjustment can be achieved using a motor that is controllable by an operator of the vehicle 110, where the motor can be used to raise or lower the measurement apparatus 130 along the line H.

The arrangement of the boom 120, the mounting pole 125 and the measurement apparatus 130 shown in FIG. 1B is given by way of example, and other arrangements and approaches are possible. Also, the vehicle 110, the boom 120 and the mounting pole 125 are shown for purposes of illustration and may not be to scale. For instance, the vehicle 110, the boom 120 and the mounting pole 125 can be configured such that they do not interfere, or damage plants of a crop being assessed. That is, the vehicle 110 and the boom 120 can be configured to have sufficient ground clearance in the planting field 140, such that they pass over plants of the crop being assessed (e.g., the maize plant 150), while the mounting pole 125 can be configured such that it does not contact the plants as the vehicle 110 moves through the field (e.g. passes to the side of a planting row).

FIG. 2 is a block diagram schematically illustrating an example measurement apparatus 200 that can be included, e.g., in the implementation of FIGS. 1A and 1B. For instance, the measurement apparatus 200 can be used to implement the measurement apparatus 130 of the arrangement 100 of FIG. 1A. In this example, the measurement apparatus 200 includes an instrumentation portion 230a and a computing device 230b. As shown in FIG. 2, the instrumentation portion 230a includes an alignment mechanism 232, a deflection mechanism 234 and a force sensor 236. In the measurement apparatus 200, the computing device 230b can communication with the instrumentation portion 230a, such as to receive electrical and/or data signals from the force sensor 236 (e.g., indicating an amount of force applied to the deflection mechanism 234 by a plant stalk) and/or to control operation of the deflection mechanism 234, such as to control rotational speed(s) of wheels included in the deflection mechanism 234 (e.g., FIGS. 3A-3B and 9A-9B), e.g., to match a velocity of a corresponding vehicle (e.g., the vehicle 110).

In some implementations, the instrumentation portion 230a can be included on (attached to) a boom of an agricultural vehicle, such as in the arrangement of the measurement apparatus 130 on the boom 120 shown in FIGS. 1A and 1B. In some implementations, the computing device 230b can be located in a driver compartment of the associated vehicle. In some implementations, the computing device 230b can be co-located with the instrumentation portion 230a. The specific arrangement of the instrumentation portion 230a and the computing device 230b will depend on the particular implementation. In the example of FIG. 2, the computing device 230b is illustrated as a laptop computer by way of example. In some implementations, the computing device 230b can be implemented using a number of different computing devices, such as a table computer, a smartphone, a notebook computer, etc. Depending on the particular implementation, a single computing device 230b can operate in conjunction with a single respective instrumentation portion 230a, or a single computing device 230b can operate in conjunction with multiple instrumentation portions 230a.

In example implementations of the measurement apparatus 200, the alignment mechanism 232 can be configured to, e.g., as result of movement of the vehicle relative to a plant stalk (or multiple plant stalks), to place a plant stalk (or the multiple plant stalks) in a known position relative to the measurement apparatus 200, such as discussed below with respect to the example implementations of FIGS. 3A-3B, 4A-4B, 8A-8B, and 9A-9B. For instance, in example implementations, the alignment mechanism 232 can include one more rigid members (e.g., steel beams) that guide stalks of plants being assessed to a known position, e.g., a position proximate to, or in contact with the deflection mechanism 234. As some examples, the alignment mechanism 232 can be implemented using a single, curved member, or implemented using multiple straight, or curved members to form a funnel shaped alignment mechanism. In some implementations, the deflection mechanism 234 can be configured to alternate a direction in which plant stalks are aligned, such as aligning consecutively aligned stalks in opposite directions.

The process of placing plant stalks in a known position can be referred to as pre-loading, where some amount of force can applied to the stalk when placing it in the known position. Such pre-loading can provide for stabilizing the stalk prior to structural measurements being acquired, such that the vibration of the stalk during structural measurement is reduced. Accordingly, such pre-loading, due to the stalk being stabilized, can reduce noise observed in associated structural measurements.

Further in example implementations of the measurement apparatus 200, the deflection mechanism 234 can be configured such that, as result of at least one of the movement of the vehicle relative to a plant stalk or rotational movement of the deflection mechanism, the plant stalk, or at least a portion of the plant stalk, is moved from the known position to a deflected position, where the deflected position is a distance from the known position. In example implementations, such as those described herein, the deflection mechanism 234 can be implemented using a vaned wheel, a cantilever beam or a plurality of rotational motorized wheels with offset rotational axes, where wheels that are on opposite sides of a plant stalk(s) being assessed counter-rotate so as to feed the stalk(s) through the wheels of the deflection mechanism 234.

Also in example implementations of the measurement apparatus 200, the force sensor 236 can be configured to determine an amount of force applied to move the plant stalk from the known position to the deflected position. In some implementations, the force sensor 236 can be integrated with (operationally coupled with, included in, etc.) the deflection mechanism 234. For instance, the force sensor 236 can be integrated in a wheel of a deflection device to facilitate structural measurements (e.g., force and/or strain measurements) associated with mechanical pressure, or mechanical force applied to the deflection mechanism 234 by the stalk.

In some implementations, strain gauges can be used on a deflection mechanism 234 implemented using a cantilever beam having known flexural rigidity characteristics, where the strain gauges can determine an amount of mechanical strain placed on the cantilever beam by a plant stalk being assessed, which can then be indicated to the computing device 230b as respective electrical or data signals. In such implementations, information obtained from multiple force sensors (e.g., strain gauges and/or load cells) can be used to determine (e.g., through mathematical calculations) the amount of force placed on the cantilever beam by the stalk (or stalks). An example of making such a determination is discussed in further detail below with respect to FIGS. 5A-7. In example implementations using a plurality of counter-rotating wheels to implement the deflection mechanism 234, respective force sensors can be included in one or more of the wheels to facilitate determining an amount of force place a plant stalk to achieve a given amount of deflection, where the amount of deflection can be determined based on an overlap between respective radii of the wheels, and spacing of the wheels from each other (e.g., distances between respective rotational plane of the wheels).

FIGS. 3A and 3B are diagrams illustrating an example implementation of the measurement apparatus 200 of FIG. 2. Specifically, FIGS. 3A and 3B schematically illustrate elements of, and operation of an example implementation of the instrumentation portion 230a of the measurement apparatus 200. A computing device, such as the computing device 230b is not shown in FIGS. 3A and 3B.

FIG. 3A illustrates a top down view of a row of plants (e.g., maize stalks) for which structural measurements are being taken for assessing flexural stiffness and/or bending strength with a measurement apparatus attached to a vehicle traveling direction T. As shown in FIG. 3A, an alignment mechanism 332, implemented using a curved member, is configured, as the vehicle moves along the row, to contact the stalks and move them into a known position. For instance, in the example of FIG. 3A, a stalk 350a is contacted with the alignment mechanism 332 and as the vehicle, and the alignment mechanism 332 move along the direction T, the stalk 350a will be aligned (moved to the right) to a known position. In this example, a stalk 350k is shown as being in the known position, e.g. at a lower end of the alignment mechanism 332 in the view of FIG. 3A.

As illustrated in FIG. 3A, the known position is proximate an integrated deflection mechanism and force sensor assembly (integrated assembly 334). In this example, the integrated assembly 334 includes a motorized wheel 334w, which can include an integrated load cell and/or can be operationally coupled with a load cell. As shown in FIG. 3A, the motorized wheel 334w can include a plurality of vanes 334v that are uniformly distributed around a perimeter of the motorized wheel 334w. As the vehicle, including the attached measurement apparatus, moves along the direction T, the plant stalks can be accepted between adjacent vanes 334v of the motorized wheel 334w, and rotation of the motorized wheel 334w can move the stalks from the known position to the deflected position, such as is depicted by a stalk 350d. In some implementations, the motorized wheel 334w can be configured to rotate in a direction 335 at a rotational speed that corresponds with a velocity of the vehicle and the attached measurement apparatus to ensure that the vanes 334v do not damage the stalks during deflection and/or exert forces on the load cell that are not related to the flexural stiffness and/or bending strength of the stalks.

In this example, as a given stalk is moved from the known position to the deflected position, the load cell of the integrated assembly 334 can determine an amount of force the stalk exerts on the motorized wheel 334w, which can be referred to as a deflection force. The deflection force can then be indicated to an associated computing device as an electrical and/or data signal. After deflection of a given stalk and the vehicle moves along the direction T, the stalk will be separated from the integrated assembly 334 and return to its natural growing position.

Referring to FIG. 3B, a view of the example of FIG. 3A, viewed from the bottom of the view in FIG. 3A along the direction T, is illustrated. That is, in the view of FIG. 3B, the direction T is into the page. As shown in FIG. 3B, the motorized wheel 334w of the integrated assembly 334 can rotate on an axis 360 at a height h, where the height h can be varied as described herein, such as with respect to FIG. 1B. For illustration purposes, the stalk 350a and the stalk 350d are shown in FIG. 3B. For purposes of clarity, other elements of FIG. 3A, such as the alignment mechanism 332 and the stalk 350k, for example, are not shown in FIG. 3B. In such implementations, an amount of deflection (e.g. a lateral distance between the known position and the deflected position) is based on a radius of the motorized wheel 334w. Based on the height h, the amount of deflection, and the deflection force, flexural stiffness of a given stalk can be determined.

In some implementations, the integrated assembly 334 can be used to determine bending strength of a plant stalk being assessed. For instance, the height h can be reduced so the motorized wheel 334w deflects the stalk closer to the ground surface and/or a radius of the motorized wheel 334w can be increased to increase an amount of force between the stalk and the integrated assembly motorized wheel 334w. In such implementations, fracture or breakage of the stalk can be identified when an amount of force on the motorized wheel 334w has a step-wise decrease as a result of the fracture. In such implementations, breaking strength can be determined based on the corresponding height h of the integrated assembly 334, an amount of deflection (deflection distance) at time of fracture, and the amount of deflection force determined (or measured) by the load cell just prior to fracture.

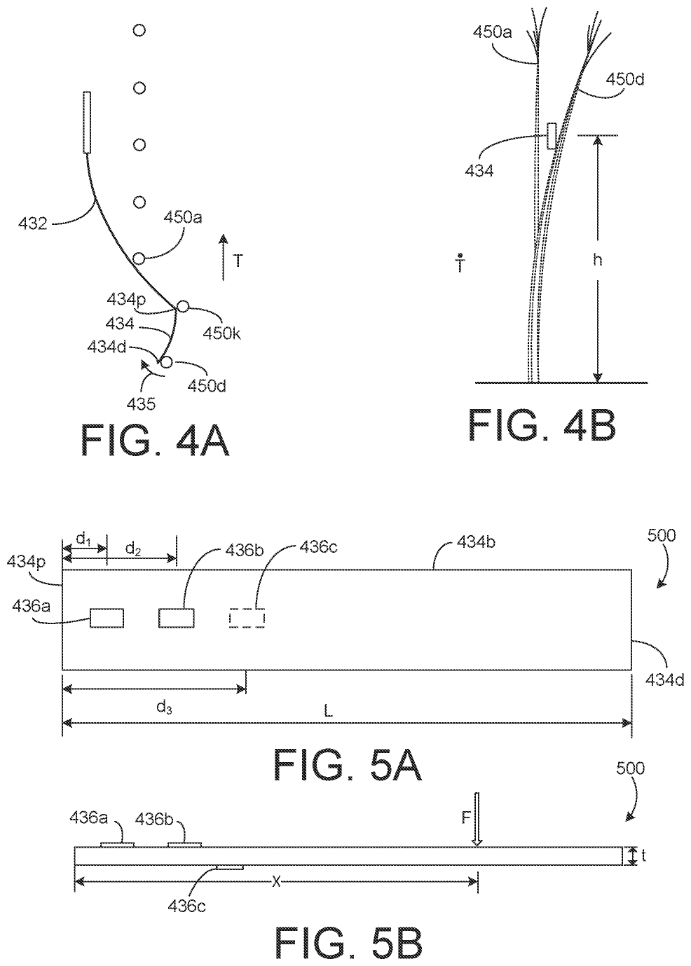

FIGS. 4A and 4B are diagrams illustrating another example implementation of the measurement apparatus of FIG. 2. Specifically, as with FIGS. 3A, 3B, FIGS. 4A and 4B schematically illustrate elements of, and operation of an example implementation of the instrumentation portion 230a of the measurement apparatus 200. Again, as with FIGS. 3A, 3B, a computing device, such as the computing device 230b, is not shown in FIGS. 4A and 4B. As with the example of FIG. 3A, FIG. 4A illustrates a top down view of a row of plants (e.g., maize stalks) for which structural measurements are being taken for assessing flexural stiffness and/or bending strength with a measurement apparatus attached to a vehicle traveling direction T.

As shown in FIG. 4A, an alignment mechanism 432, implemented using a curved member, is configured, as the vehicle moves along the row, to contact the stalks and move them into a known position. For instance, in the example of FIG. 4A, a stalk 450a is contacted with the alignment mechanism 432 and as the vehicle, and the alignment mechanism 432 move along the direction T, the stalk 450a will be aligned (moved to the right) to a known position. In this example, a stalk 450k is shown as being in the known position, e.g. at a lower end of the alignment mechanism 432 in the view of FIG. 4A.

As illustrated in FIG. 4A, the known position is proximate an integrated deflection mechanism and force sensor assembly (integrated assembly 434). In this example, the integrated assembly 434 includes a cantilever beam that can have one or more strain gauges (not shown in FIGS. 4A and 4B) that are disposed on the cantilever beam, such as in the example of FIGS. 5A and 5B. As shown in FIG. 4A, a proximal end 434p of the cantilever beam of the integrated assembly 434 can be coupled with the alignment mechanism 432, while a distal end 434d of the cantilever beam can be free moving. Such an arrangement allows the cantilever beam to be deflected from a biased position, e.g., along line 435, by stalks of crop material being assessed.

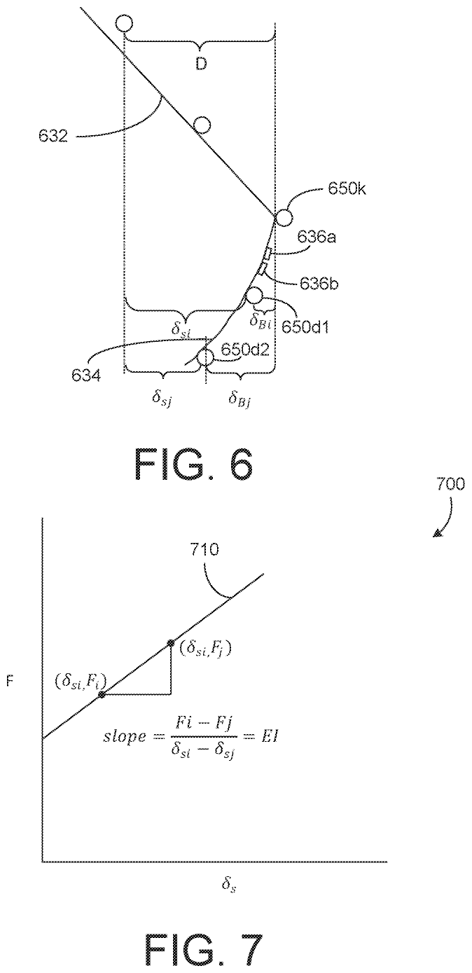

In some implementations, the proximal end 434p can be coupled with the alignment mechanism 432 in a fixed position relative to the alignment mechanism 432. In other implementations, the proximal end 434p of the cantilever beam can be affixed to the alignment mechanism 432 via a torsional spring that includes an integrated torsional load cell, and/or is operatively coupled with a torsional load cell. In such implementations, measurements obtained using the one or more strain gauges of the integrated assembly 434 and/or by the torsional load cell can be used to determine flexural stiffness and/or bending strength of assessed crop material, such as using the approach (or a similar approach) described below with respect to, at least, FIGS. 6 and 7 (using an implementation of the example beam sensor of FIGS. 5A and 5B).

In this example, while a given stalk is moving from the known position (stalk 450k) to the deflected position (stalk 450d), the one or more strain gauges and/or the torsional load cell can obtain measurements that can be used to determine an amount of force the stalk exerts on the cantilever beam of the integrated assembly 434 (e.g., at a given position along the cantilever beam), which can be referred to as a deflection force. After deflection of a given stalk and the vehicle moves along the direction T, the cantilever beam of the integrated assembly 434 will pass the stalk, allowing the stalk to separate from the integrated assembly 434 and return to its natural growing position.

Referring to FIG. 4B, a view of the example of FIG. 4A, viewed from the bottom of the view in FIG. 4A along the direction T, is illustrated. That is, in the view of FIG. 4B, the direction T is into the page. As shown in FIG. 4B, the cantilever beam of the integrated assembly 434 can be at a height h, where the height h can be varied as described herein, such as with respect to FIG. 1B. For illustration purposes, the stalk 450a and the stalk 450d are shown in FIG. 4B. For purposes of clarity, other elements of FIG. 4A, such as the alignment mechanism 432 and the stalk 450k, for example, are not shown in FIG. 4B. In such implementations, an amount of deflection (e.g. a lateral distance between the known position and the deflected position) is based on an amount of deflection of the cantilever beam of the integrated assembly 434, which can be calculated as described below. Based on the height h, the amount of deflection, and the determined deflection force, flexural stiffness of a given stalk can be determined.

In some implementations, the integrated assembly 434 can be used to determine bending strength of a plant stalk being assessed. For instance, the height h can be reduced so the cantilever beam of the integrated assembly 434 deflects the stalk closer to the ground surface, a length of the cantilever beam can be increased, and/or a stiffness of the cantilever beam can be increased to increase an amount of force applied to the stalk by the cantilever beam during deflection. In such implementations, fracture or breakage of the stalk can be identified when an amount of force between the cantilever beam and the stalk (indicated by measurements from the one or more strain gauges and/or the torsional load cell) has a step-wise decrease as a result of the fracture. In such implementations, breaking strength can be determined based on the corresponding height h of the integrated assembly 434, an amount of deflection (deflection distance) at time of fracture, and the amount of deflection force (determined, calculated and/or measured) just prior to fracture.

FIGS. 5A and 5B are diagrams are diagrams illustrating an example cantilever beam sensor 500 that can be included in the measurement apparatus of FIGS. 4A and 4B. That is, the cantilever beam sensor 500 can be included in the measurement apparatus of FIGS. 4A and 4B. In this example, the cantilever beam sensor 500, as shown in FIGS. 5A and 5B, can include a cantilever beam 434b with known physical properties, such as length L, thickness t, Young's Modulus E, etc. As shown in FIGS. 4A and 4B, the cantilever beam sensor 500 can have a plurality of strain gauges disposed on the cantilever beam 434b. In this example, a strain gauge 436a and a strain gauge 436b are disposed on a first side (surface) of the cantilever beam 434b, while a strain gauge 436c is disposed on a second side (surface) of the cantilever beam 434b, where the second side is opposite the first side.

In this example, a center of the strain gauge 436a is located at a distance d1 from the proximal end 434p of the cantilever beam 434b, a center of the strain gauge 436b is located at a distance d2 from the proximal end 434p of the cantilever beam 434b, and a center of the strain gauge 436c is located at a distance d3 from the proximal end 434p of the cantilever beam 434b, where d2 is greater than d1 and d3 is greater than d2. Such distances can be used, in combination with measurements made by the strain gauges, to determine a location (i.e., a distance X from the proximal end 434p) and an amplitude of a force (represented as F) applied to the cantilever beam 434b, e.g., by a stalk (or stalks) being assessed. A deflection distance can also be determined based on the physical properties of the cantilever beam 434b, and measurements from the strain gauges and/or a corresponding torsional spring used to couple the cantilever beam 434b with an alignment mechanism, such as the alignment mechanism 432 in FIG. 4A.

The placement of the strain gauges as shown in FIGS. 5A and 5B is given by way of example, and other arrangements are possible. For instance, the strain gauge 436c could be placed at the distance d1 from the 434p (e.g., on an opposite surface of the distal end 434d from the strain gauge 436a), or additional strain gauges could be disposed on the cantilever beam 434b at the distances shown, at other distances, and/or on either side, or both sides of the cantilever beam 434b (e.g., the top surface and the bottom surface as shown in FIG. 5B). Use of such strain gauge arrangements can allow for greater measurement sensitivity, as they can allow for making redundant measurements, and/or multiple measurements for an applied force at different locations along the distal end 434d, which can improve accuracy and/or reduce measurement noise (e.g., by averaging corresponding measurements).

Still referring to FIGS. 5A and 5B, the following discussion provides an example approach for determining, using measurements from the strain gauge 436a and the strain gauge 436b, where strain observed at each strain gauge is represented by a respective voltage. In this example, respective strain measurements for each of the strain gauges are taken at a same time and, accordingly, can correspond to an exact same applied force on the cantilever beam 434b. For purposes of discussion, measurements from the strain gauge 436c and/or an associated torsional load cell are not considered in the discussed with respect to the following example. However, similar approaches and calculations can be applied to such measurements (separate from, and/or in combination with stress measurements from the strain gauges 436a and 436b).

In this example, respective measurement signals of the strain gauge 436a and the strain gauge 436b can be a voltage signal V that have respective linear relationships with observed strain ε, and having respective slope constants k. Accordingly, in this example, V₁ and k₁ represent the voltage and slope constant for the strain gauge 436a, while V₂ and k₂ represent the voltage and slope constant for the strain gauge 436b. In example implementations, the constants k₁ and k₂ are found through calibration of the cantilever beam sensor 500. Based on the foregoing, strain for the each of the strain gauge 436a and the strain gauge 436b can be respectively given by Equations 1 and 2 as:

$${V}_{1} {k}_{1} = {ε}_{1} \qquad ( 1 )$$

$${V}_{2} {k}_{2} = {ε}_{2} \qquad ( 2 )$$

Strain can be determined from stress using Hooke's law as σ=Eε, where σ is stress and E is the Young's Modulus of the cantilever beam 434b. In a cantilever beam, such as the cantilever beam 434b, stress depends upon geometry of the beam and a respective moment experienced by an associated strain gauge.

Further, respective stress observed for the strain gauge 436a (index i=1) and the strain gauge 436b (index i=2) can each be given by Equation 3 as:

$${σ}_{i} = \frac{{M}_{i} ( t / 2 )}{I} \qquad ( 3 )$$

where t is the thickness of the beam (as shown in FIG. 5B), Mᵢ is the respective moment at each strain gauge, and I is the area moment of inertia of the beam, where I is a geometric property of the beam.

Therefore, the moment at each of the strain gauges can be respectively represented by Equations 4 and 5 as:

$${M}_{1} = F ( x - {d}_{1} ) \qquad ( 4 )$$

$${M}_{2} = F ( x - {d}_{2} ) \qquad ( 5 )$$

Where d₁ is the distance from the proximal end 434p of the cantilever beam 434b to the middle of the strain gauge 436a, and d2 is the distance from the proximal end 434p of the cantilever beam 434b to the middle of the strain gauge 436b (e.g., as shown in FIG. 5A). In Equations 4 and 5, x is the distance of the applied force on the cantilever beam 434b from its proximal end 434p, and F is the magnitude of the applied force.

Using algebraic operations and value substitution, F can be expressed in terms of Vᵢ kᵢ E, I, t, and dᵢ. As the exact same force, at the exact same distance x from the proximal end 434p is being measured by both strain gauges in this example, noting the value of F is the same for both strain gauges. F for either strain gauge can generally be arrived at using the sequence of Equations 6 to 9 below:

$$σ = \frac{M ( t / 2 )}{I} \qquad ( 6 )$$

$$E ε = \frac{F ( x - {d}_{i} ) ( t / 2 )}{I} \qquad ( 7 )$$

$${V}_{i} {k}_{i} = \frac{F ( x - {d}_{i} ) ( t / 2 )}{EI} \qquad ( 8 )$$

$${F}_{i} = \frac{{V}_{i} {k}_{i} EI}{( x - {d}_{i} ) ( t / 2 )} \qquad ( 9 )$$

As noted above, the magnitude of the force being measure at each of the strain gauges 436a and 436b is the same, which is given by Equation 10 as:

$${F}_{1} = {F}_{2} \qquad ( 10 )$$

Substituting Equation 9 for each strain gauge into Equation 10 provides Equation 11 below, which allows for determining the location on the cantilever beam 434b that the force is applied, i.e., the distance x from the proximal end 434p. Equation 11, which can be reduced to Equation 12, where Equation 12 can then be used to solve for x as Equation 13.

$$\frac{{V}_{1} {k}_{1} EI}{( x - {d}_{1} ) ( t / 2 )} = \frac{{V}_{2} {k}_{2} EI}{( x - {d}_{2} ) ( t / 2 )} \qquad ( 11 )$$

$$\frac{{V}_{1} {k}_{1}}{{V}_{2} {k}_{2}} = \frac{x - {d}_{1}}{x - {d}_{2}} \qquad ( 12 )$$

$$x = \frac{{V}_{1} {k}_{1} {d}_{2} - {V}_{2} {k}_{2} {d}_{1}}{{V}_{1} {k}_{1} - {V}_{2} {k}_{2}} \qquad ( 13 )$$

Now that a formulation for x is known, x can be substituted back into Equation 9 to obtain the value of the applied force F. The deflection of the beam δ_{B} can then found by using the standard equation for deflection of a cantilever beam using Equation 14:

$${δ}_{B} = \frac{{Fx}^{3}}{3 EI} \qquad ( 14 )$$

In some implementations, using the cantilever beam sensor 500 to determine an applied force, a location of the applied force and associated beam deformation can include the following operations:
1. Calibrate the strain gauges to obtain k₁ and k₂
2. Apply a force at any point on the beam and measure voltages V₁ and V₂.
3. Solve for the magnitude of the applied force using Equation 9.
4. Solve for the location of the applied force on the beam using Equation 13.
5. Solve for the beam deformation using Equation 14.
   While the forgoing example uses equations of small-displacement mechanics, in some implementations, alternative formulations using large-displacement mechanics can also be used for to arrive at the same, or similar results.

FIG. 6 is a diagram illustrating assessment of flexural stiffness and/or bending strength of crop material with an implementation of the measurement apparatus of FIGS. 4A, 4B and/or the cantilever beam sensor 500 of FIGS. 5A and 5B. In the example of FIG. 6, animation of a single plant stalk is shown as it moves along the measurement apparatus, e.g., due to motion of an associated vehicle. That is, the stalk can be aligned to a known position 650k using an alignment mechanism 632. As an associated vehicle continues forward motion long the direction T, the stalk will move along a cantilever beam 634 having a strain gauge 636a and a strain gauge 636b disposed thereon, such as with the strain gauges 436a and 436b described above with respect to FIGS. 5A and 5B.

The flexural stiffness of the stalk will apply force to the cantilever beam 634 (e.g., at a height h along the stalk, such as shown in FIG. 4B). In FIG. 6, the indices i and j indicate two respective locations of the plant stalk as it moves along the cantilever beam 634. In this example, a relationship between beam deformation (δ_{B}), stalk deformation (δ_{S}), and initial displacement of the stalk (D), as shown in FIG. 6, is given by Equation 15 as:

$${δ}_{S} = D - {δ}_{B} \qquad ( 15 )$$

Again, as noted above, stiffness can be defined as the slope of an associated force deformation curve. Although Equation 15 does not provide for solving for an absolute displacement D of the stalk, it does allow for determining a change in force and a change in deflection of the plant stalk between locations i and j, where δ_{Bi} is the deformation (deflection) of the cantilever beam 634 at location i, δ_{Si} is the deformation (deflection) of the stalk at location i, δ_{Bj} is the deformation (deflection) of the cantilever beam 634 at location j, δ_{Sj} is the deformation (deflection) of the stalk at location j. With this information, the force/deformation slope can be obtained, such as illustrated by FIG. 7 below.

FIG. 7 is a graph illustrating a force deformation curve 710 corresponding with the assessment of FIG. 6, which includes a formula for the slope of the force deformation curve 710. As shown in FIG. 7, by applying Equation 15 at two locations along the cantilever beam 634 (i and j), respective equations relating stalk deformation and beam deformation δ_{S} with beam deformation δ_{B} for each location i and j can be determined. By subtracting the j equation from the i equation, we can eliminate the unknown D and arrive at Equation 16:

$$\frac{\begin{matrix}{δ}_{Si} = D - {δ}_{Bi} \\ - {δ}_{Sj} = D - {δ}_{Bj}\end{matrix}}{{δ}_{Si} - {δ}_{Sj} = {δ}_{Bj} - {δ}_{Bi}} \qquad ( 16 )$$

By definition, the formula for stiffness of the stalk (EI) is given by Equation 17 as:

$$EI = \frac{{Fh}^{3}}{{δ}_{s}} \qquad ( 17 )$$

where h is the height perpendicular to the ground at which the beam is pressing against the stalk (e.g., FIG. 4B). The term F/δ_{S} represents a corresponding slope of the associated force/deformation curve. By substituting the slope equation shown in FIG. 7 into Equation 17, EI can be determined by Equation 18 as:

$$EI = \frac{( {F}_{i} - {F}_{j} )}{( {δ}_{Bj} - {δ}_{Bi} )} ( \frac{{h}^{3}}{3} ) \qquad ( 18 )$$

which can be used to solve for the stiffness of the stalk (EI).

In this example, only two data points (i and j) are used for the purposes of calculation, flexural stiffness. In some implementation, a vehicle including measurement device such as those of FIGS. 4A-6, voltages can be continuously measured by associated strain gauges as each stalk slides along a cantilever beam (e.g., the cantilever beam 434b, the cantilever beam 634, etc., which can provide numerous locations of measurement along the associated cantilever beam. Any two data points (e.g., as long as both points are distal of a distal-most strain gauge providing a measurement for one of the locations) can be used to calculate stalk flexural stiffness. For instance, by combining many different combinations of data pairs, stalk stiffness can be calculated multiple times over the course of a stalk passing over a cantilever beam sensor. An average of these values can provides an estimate of stalk flexural stiffness that is less sensitive to measurement error and/or measurement noise.

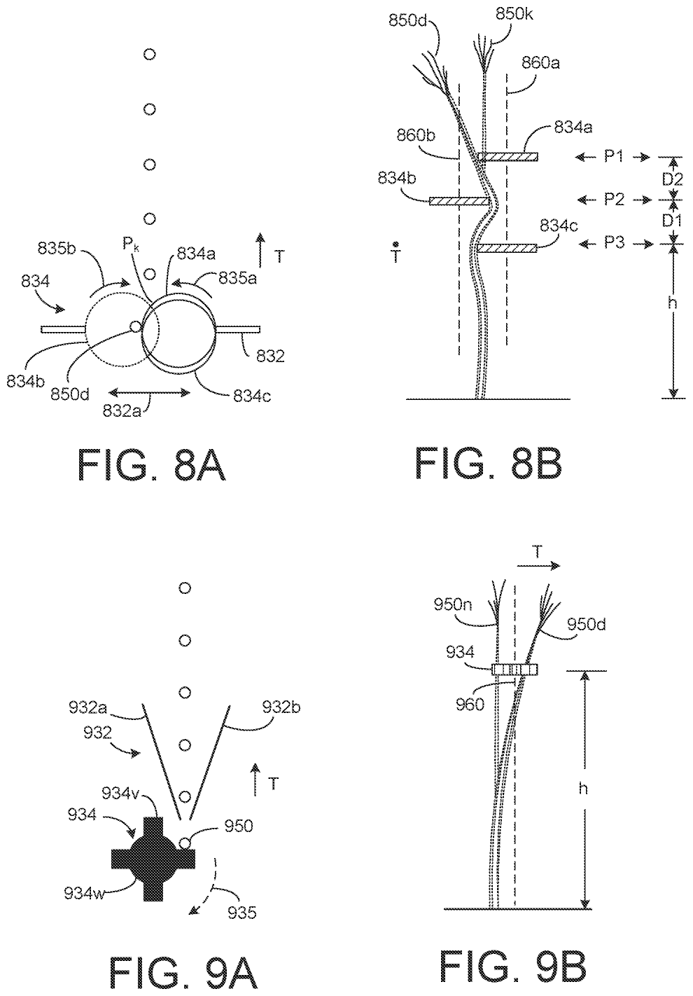

FIGS. 8A and 8B are diagrams illustrating yet another example implementation of the measurement apparatus of FIG. 2. Specifically, as with FIGS. 3A, 3B, and FIGS. 4A and 4B, FIGS. 8A and 8B schematically illustrate elements of, and operation of an example implementation of the instrumentation portion 230a of the measurement apparatus 200. Again, as with FIGS. 3A, 3B, 4A and 4B, a computing device, such as the computing device 230b, is not shown in FIGS. 8A and 8B. As with the examples of FIGS. 3A and 4A, FIG. 8A illustrates a top down view of a row of plants (e.g., maize stalks) for which structural measurements are being taken for assessing flexural stiffness and/or bending strength with a measurement apparatus attached to a vehicle traveling direction T.

As shown in FIGS. 8A and 8B, in this example, an integrated deflection mechanism and force sensor assembly (integrated assembly 834) includes a plurality of overlapping motorized wheels. For instance, the integrated assembly 834 of this example includes a motorized wheel 834a that rotates on axis 860a in a plane P1, a motorized wheel 834b that rotates on an axis 860b in a plane P2, and a motorized wheel 834c that rotates on the axis 860a in a plane P3.

In this example, as shown in FIG. 8B, the axis 860b is spaced from and parallel to the axis 860a. Also, the axis 860a and the axis 860b can be substantially perpendicular to a ground surface (e.g., planting field), such that the axis 860a and the axis 860b are substantially parallel a longitudinal axis of a stalk being evaluated. Here, because the ground surface of the planting field may be irregular, and because plants stalks can be longitudinally irregular and may not be perfectly vertical with respect to a planting surface, the reference to substantially perpendicular and substantially parallel are used to account for, and in view of such variations and irregularities.

In some implementations, the motorized wheels of the integrated assembly 834 can all have a same radius. In other implementations, the motorized wheel 834a and the motorized wheel 834b can both have a same radius, while the motorized wheel 834c can have a different radius. Regardless of the respective radii, to achieve overlap of the motorized wheels (e.g., to facilitate stalk deflection), a sum of the radius of the radius of the motorized wheel 834a (or a radius of the motorized wheel 834c) plus a radius of the motorized wheel 834b should be larger than a spacing between the axis 860a and the axis 860b.

In this example, respective force sensors (load cells) for measuring deflection forces can be included in one, two or all three of motorized wheels of the integrated assembly 834. As shown in FIG. 8B, the motorized wheel 834b is spaced from the motorized wheel 834a by a distance D1, and the motorized wheel 834c is spaced from the motorized wheel 834b by a distance D2. That is, the planes (rotational planes) P1, P2 and P3 are different planes that are spaced from each other. The distance D1 and the distance D2, in combination with an amount of overlap of the radii of the motorized wheels can define a deflection distance for stalks that are evaluated using the integrated assembly 834.

As shown in FIG. 8A, the motorized wheel 834a and the motorized wheel 834c rotate in a direction 835a, while the motorized wheel 834b rotates in a direction 835b, which is counter to the direction 835a. Said another way, the motorized wheel 834b counter-rotates with respect to the motorized wheel 834a and the motorized wheel 834c. This counter-rotation can facilitate moving a plant stalks that is being assessed from a known position Pₖ to a deflected position, where the deflected position is indicated by a stalk 850d. As with the example of FIGS. 3A and 3B, the rotational speeds of the motorized wheels of the integrated assembly 834 can correspond with a velocity of the vehicle and the attached measurement apparatus, so as to prevent damage to stalks being evaluated and/or to prevent forces on the load cell(s) of the integrated assembly 834 that are not related to the flexural stiffness and/or bending strength of the stalks.

As shown in FIG. 8A, in this example, the measurement apparatus includes an alignment mechanism 832, which can be implemented using a linear bearing assembly. For instance, in the example of FIG. 8A, when a stalk contacts at least one the motorized wheel 834a, the motorized wheel 834b, or the motorized wheel 834c, the alignment mechanism 832, friction between the rotating wheel(s) and the stalk will cause the integrated assembly 834 to move laterally (orthogonally to the direction T) along line 832a, which will, in combination with movement of the vehicle along the direction T, move the stalk to the known position Pₖ. Once the stalk is in the known position Pₖ, the counter-rotating wheels of the integrated assembly 834 will pull the stalk into a deflected position, such as depicted by the stalk 850d in FIGS. 8A and 8B.

In this example, as a given stalk is moved from the known position Pₖ to the deflected position, the load cell(s) of the integrated assembly 834 can determine a respective amount of force the stalk exerts each of the motorized wheels including a load cell, which, as noted above, can be referred to as deflection forces. These deflection forces can then be indicated to an associated computing device as an electrical and/or data signal. After deflection of a given stalk, rotation of the wheels will cause the stalk to be discharged from the integrated assembly 834, and as the vehicle moves along the direction T, the stalk will be separated from the integrated assembly 834 and return to its natural growing position.

Referring to FIG. 8B, a view of the example of FIG. 8A, viewed from the bottom of the view in FIG. 8A along the direction T, is illustrated. That is, in the view of FIG. 8B, the direction T is into the page. For illustration purposes, the stalk 850d and a stalk 850a (e.g., in the known position Pₖ) are shown in FIG. 8B. For purposes of clarity, other elements of FIG. 8A, such as the alignment mechanism 832 are not shown in FIG. 8B. In this example, based on the distances D1 and D2, the determined amount of deflection (as discussed above), and the determined deflection force(s), flexural stiffness of a given stalk can be determined.

In some implementations, the integrated assembly 834 can be used to determine bending strength of a plant stalk being assessed. For instance, a height h of the integrated assembly 834 can be reduced so the motorized wheels deflect the stalk closer to the ground surface, and/or an overlap of the motorized wheels can be increased to increase an amount of force placed on the stalk during deflection. In such implementations, fracture or breakage of the stalk can be identified when an amount of force detected by the load cell(s) of the integrated assembly 334 has (or have) a step-wise decrease as a result of the fracture. In such implementations, breaking strength can be determined based on the distances D1 and D2, an amount of deflection (deflection distance) at time of fracture, and respective amount(s) of deflection force determined or measured by the load cell(s) just prior to fracture.

FIGS. 9A and 9B are diagrams illustrating still another example implementation of the measurement apparatus of FIG. 2. Specifically, as with FIGs. FIGS. 3A and 3B, FIGS. 4A and 4B, and FIGS, 8A and 8B, FIGS. 9A and 9B schematically illustrate elements of, and operation of an example implementation of the instrumentation portion 230a of the measurement apparatus 200. Also, as with FIGS. 3A and 3B, FIGS. 4A and 4B, and FIGS, 8A and 8B, a computing device, such as the computing device 230b, is not shown in FIGS. 9A and 9B.

FIG. 9A illustrates a top down view of a row of plants (e.g., maize stalks) for which structural measurements are being taken for assessing flexural stiffness and/or bending strength with a measurement apparatus attached to a vehicle traveling direction T. As shown in FIG. 9A, an alignment mechanism 932, implemented using a member 932a and a member 932b to define a funnel shape, is configured, as the vehicle moves along the row, to direct the stalks into a known position, such as at the opening (narrow open end) in the funnel shape of the alignment mechanism 932.

As illustrated in FIG. 9A, the known position is proximate an integrated deflection mechanism and force sensor assembly (integrated assembly 934). In this example, the integrated assembly 934 includes a wheel 934w, which can include an integrated load cell and/or can be operationally coupled with a load cell. As shown in FIG. 9A, the wheel 934w can include a plurality (four) vanes 934v that are uniformly distributed around a perimeter of the wheel 934w, where the vanes 934v form a cross-shape.

As the vehicle, including the attached measurement apparatus, moves along the direction T, aligned plant stalks (from the funnel opening of the alignment mechanism 932) can contact a surface one of the vanes 934v of the wheel 934w, which can be locked in the position shown in FIG. 9A when a force applied to the contacted vane 934v by the stalk is less than a threshold value. In this example, when the threshold force is reached or exceeded, the wheel 934w can be released and rotate 90 degrees, which allows the current stalk to be released back to its natural growing position, as well as positions a next vane 934v to accept a next stalk for evaluation. In some implementations, a mechanical torque limiter can be used to control locking, unlocking and rotation of the wheel 934w. In other implementations, a solenoid can be used to positionally lock and unlock the wheel 934w, e.g., based on an amount of force detected by the load cell of the integrated assembly 934 and a threshold force value.

In this example, an amount of deflection (deflection distance) can be determined based on a distance the associated vehicle travels between a time that the stalk contacts a vane 934v of the wheel 934w until the threshold force is reached, and the of the wheel 934w is positionally released (e.g., by use of a solenoid or a torque limiter) and rotated to release the stalk being evaluated. In implementations using a mechanical torque limiter, the point of release can be determined by a step-wise drop in force applied to the vane 934v of the wheel 934w when the wheel 934w rotates and the stalk is released.

Referring to FIG. 9B, a view of the example of FIG. 9A, viewed from the right of the view in FIG. 9A, is illustrated. That is, in the view of FIG. 9B, the direction T is left-to-right on the page. As shown in FIG. 9B, the wheel 934w of the integrated assembly 934 can be mounted on and/or rotate on an axis 960 at a height h, where the height h can be varied as described herein, such as with respect to FIG. 1B. For illustration purposes, FIG. 9B shows a stalk 950d (in a deflected position) and a stalk 950n, which depicts the stalk 950d in its natural growing position, prior to and/or after its deflection. For purposes of clarity, the alignment mechanism 932 shown in FIG. 9A is not shown in FIG. 9B. In such implementations, as noted above, an amount of deflection (e.g. a deflection distance is based on a distance the associate vehicle travels while the corresponding stalk is in contact with a vane 934v of the wheel 934w. Based on the height h, the amount of deflection (deflection), and the deflection force (e.g., the force threshold), flexural stiffness of a given stalk can be determined.

In some implementations, the integrated assembly 934 can be used to determine bending strength of a plant stalk being assessed. For instance, the height h can be reduced so the vanes 934v of the wheel 934w deflect the stalks closer to the ground surface and/or the wheel 934w can remain positionally fixed regardless of the amount of force applied to the wheel 934w by the stalk. In such implementations, fracture or breakage of the stalk can be identified when an amount of force applied to the wheel 934w has a step-wise decrease as a result of the fracture. In such implementations, breaking strength can be determined based on the corresponding height h of the integrated assembly 934, an amount of deflection (deflection distance) at time of fracture, and the amount of deflection force determined (or measured) by the load cell just prior to fracture.

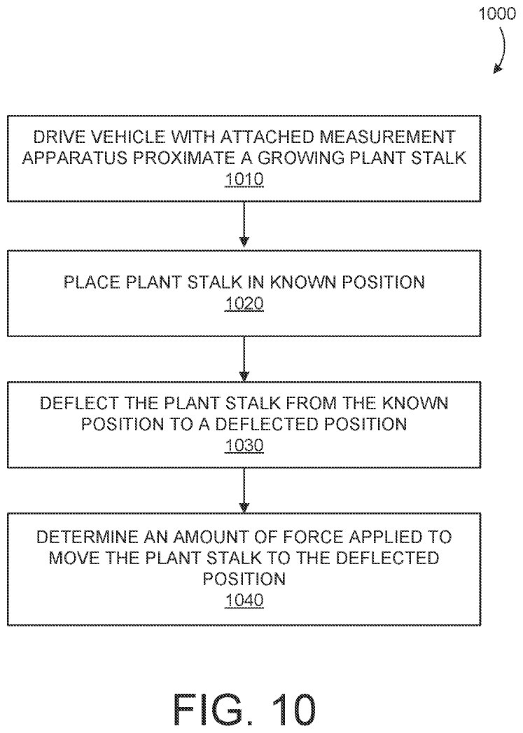

FIG. 10 is a flowchart illustrating an example method 1000 for assessment of flexural stiffness and/or bending strength of growing plant material, such as stalked plants of an agricultural crop. In some implementations, the method 1000 can be performed using the apparatus and approached described herein. For instance, the method 1000 can be performed using the arrangement 100 of FIGS. 1A and 1B, where the measurement apparatus 130 can be implemented using the examples of FIGS. 3A-3B, 4A-4B, 8A-8B, and/or 9A-9B. In some implementations, other measurement apparatus could be used to implement the method 1000. More or fewer operations than shown in FIG. 10 can be performed. Two or more operations can be performed in a different order, or at a same time unless otherwise indicated.

As shown in FIG. 10, at block 1010, the method 1000 includes driving a vehicle having a measurement apparatus attached thereto proximate to a growing plant stalk, such as in the example illustrated in FIG. 1B. At block 1020, the method 1000 includes, as a result of movement of the vehicle, an alignment mechanism placing a plant stalk in a known position relative to the measurement apparatus, such as described, for example, with respect to FIGS. 3A, 4A, 8A and 9B. At block 1030, the method 1000 includes moving, with a deflection mechanism as a result of at least one of the movement of the vehicle relative to the plant stalk (e.g., FIGS. 4A and 9A) or rotational movement of the deflection mechanism (e.g., FIGS. 3A and 8A), the plant stalk from the known position to a deflected position, where the deflected position is a distance from the known position. At block 1040, which can be performed contemporaneously with the operation of block 1030, the method 1000 includes determining, with a force sensor, an amount of force applied to move the plant stalk from the known position to the deflected position (e.g., based on respective electrical signals from one more load cells and/or strain gauges). Flexural stiffness and/or bending strength (e.g., in Newton meters-squared (Nm²) can then be determined from the determined amount of force and the distance between the known position and the deflected position. As discussed with respect to the example implementations describe herein, the alignment mechanism, the deflection mechanism and the force sensor being included in the measurement apparatus.

In the foregoing disclosure, it will be understood that when an element, such as a layer, a region, or a substrate, is referred to as being on, connected to, electrically connected to, coupled to, or electrically coupled to another element, it may be directly on, connected or coupled to the other element, or one or more intervening elements may be present. In contrast, when an element is referred to as being directly on, directly connected to or directly coupled to another element or layer, there are no intervening elements or layers present. Although the terms directly on, directly connected to, or directly coupled to may not be used throughout the detailed description, elements that are shown as being directly on, directly connected or directly coupled can be referred to as such. The claims of the application may be amended to recite exemplary relationships described in the specification or shown in the figures.

As used in this specification, a singular form may, unless definitely indicating a particular case in terms of the context, include a plural form. Spatially relative terms (e.g., over, above, upper, under, beneath, below, lower, and so forth) are intended to encompass different orientations of the device in use or operation in addition to the orientation depicted in the figures. In some implementations, the relative terms above and below can, respectively, include vertically above and vertically below. In some implementations, the term adjacent can include laterally adjacent to or horizontally adjacent to.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. An apparatus configured for attachment to a vehicle, the apparatus comprising:
an alignment mechanism configured, as result of movement of the vehicle relative to a plant stalk, to place the plant stalk in a known position relative to the apparatus;
a deflection mechanism configured, as result of at least one of the movement of the vehicle relative to the plant stalk or rotational movement of the deflection mechanism, to move the plant stalk from the known position to a deflected position, the deflected position being a distance from the known position; and
a force sensor configured to determine an amount of force applied to move the plant stalk from the known position to the deflected position.

2. The apparatus of claim 1, wherein the apparatus is configured such that a height of the apparatus from a growing surface of the plant stalk is adjustable.

3. The apparatus of claim 1, wherein the force sensor includes a load cell configured to provide an electrical signal indicating the amount of force applied.

4. The apparatus of claim 1, wherein the deflection mechanism includes a wheel including a plurality of radial vanes uniformly disposed around a perimeter of the wheel.

5. The apparatus of claim 4, wherein the force sensor is operationally coupled with the wheel.

6. The apparatus of claim 4, wherein the wheel is motorized and configured to rotate at a rotational speed corresponding with a velocity of the vehicle.

7. The apparatus of claim 4, wherein:
the plurality of radial vanes includes four vanes forming a cross shape; and
the wheel is configured to:
be rotationally fixed when the amount of force applied determined by the force sensor is below a threshold value; and
in response to the amount of force applied determined by the force sensor reaching or exceeding the threshold value, rotate ninety degrees.

8. The apparatus of claim 1, wherein the alignment mechanism includes at least one rigid member.

9. The apparatus of claim 8, wherein:
the deflection mechanism includes:
a cantilever beam having a proximal end coupled to an end of a rigid member of the at least one rigid member in a fixed position relative to the rigid beam, a distal end of the cantilever beam being free moving; and
the force sensor includes:
a first strain gauge disposed on the cantilever beam at a first distance from the proximal end; and
a second strain gauge disposed on the cantilever beam at a second distance from the proximal end, the second distance being greater than the first distance.

10. The apparatus of claim 9, wherein:
the first strain gauge is disposed on a first surface of the cantilever beam; and
the second strain gauge is disposed on a second surface of the cantilever beam, the second surface being opposite the first surface.

11. The apparatus of claim 9, wherein the first strain gauge and the second strain gauge are disposed on a same surface of the cantilever beam.

12. The apparatus of claim 9, further including at least one additional strain gauge disposed on the cantilever beam, a third strain gauge of the at least one additional strain gauge being disposed at a third distance from the proximal end, the third distance being greater than or equal to the first distance.

13. The apparatus of claim 8, wherein:
the deflection mechanism includes:
a cantilever beam having a proximal end coupled to an end of a rigid member of the at least one rigid members via a torsional spring, a distal end of the cantilever beam being free moving; and
the force sensor includes:
a torsional load cell operationally coupled with the torsional spring;
a strain gauge disposed on the cantilever beam at a distance from the proximal end.

14. The apparatus of claim 13, wherein the strain gauge is a first strain gauge and the distance is a first distance, the force sensor further including a second strain gauge disposed on the cantilever beam at a second distance from the proximal end, the second distance being greater than the first distance.

15. The apparatus of claim 1, wherein:
the deflection mechanism includes:
a first wheel of a first radius being rotationally mounted on a first axis, the first wheel defining a first rotational plane; and
a second wheel of a second radius being rotationally mounted on a second axis, the second wheel defining a second rotational plane that different from the first rotational plane; and
a third wheel of the first radius being rotationally mounted on the first axis, the third wheel defining a third rotational plane that is different from the first rotational plane and the second rotational plane,
the second axis being spaced from and parallel to the first axis,
the first axis and the second axis being substantially parallel to a longitudinal axis of the plant stalk,
the second rotational plane being disposed between the first rotational plane and the third rotational plane, and
the first radius plus the second radius being greater than the spacing between the first axis and the second axis; and
the force sensor is operatively coupled with one of the first wheel, the second wheel, or the third wheel.

16. The apparatus of claim 15, wherein first wheel, the second wheel and the third wheel are rotationally motorized such that the second wheel counter-rotates with respect to the first wheel and third wheel.

17. The apparatus of claim 15, wherein the force sensor is a first force sensor operationally coupled with the second wheel, the apparatus further comprising:
a second force sensor operationally coupled with the first wheel.

18. The apparatus of claim 17, further comprising a third force sensor operatively coupled with the third wheel.

19. The apparatus of claim 15, wherein the alignment mechanism includes a linear bearing assembly configured to allow the deflection mechanism to move laterally along a line that is orthogonal to a direction of travel of the vehicle.

20. A method comprising:
driving a vehicle having a measurement apparatus attached thereto proximate to a growing plant stalk;
as a result of movement of the vehicle, an alignment mechanism placing a plant stalk in a known position relative to the measurement apparatus;
moving with a deflection mechanism, as a result of at least one of the movement of the vehicle relative to the plant stalk or rotational movement of the deflection mechanism, the plant stalk from the known position to a deflected position, the deflected position being a distance from the known position; and
determining, with a force sensor, an amount of force applied to move the plant stalk from the known position to the deflected position,
the alignment mechanism, the deflection mechanism and the force sensor being included in the measurement apparatus.

21. The method of claim 20, wherein determining the amount of force applied includes at least one of:
receiving an electric signal from at least one load cell; or
receiving an electric signal from at least one strain gauge.
